(19) United States
(12) Patent Application Publication
(10) Pub. No.: US 2008/0108912 A1
(43) Pub. Date: May 8, 2008

(54) SYSTEM AND METHOD FOR MEASUREMENT OF CLINICAL PARAMETERS OF THE KNEE FOR USE DURING KNEE REPLACEMENT SURGERY

(75) Inventor: Laurent J. Node-Langlois, Boston, MA (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/615,278

(22) Filed: Dec. 22, 2006

Related U.S. Application Data
(60) Provisional application No. 60/864,748, filed on Nov. 7, 2006.

(51) Int. Cl. A61B 5/00

(52) U.S. Cl. 600/587

(57) ABSTRACT

A system and method for measuring biomechanical parameters of a knee prior to total knee replacement (TKR) surgery includes a plurality of microsensors removably attached to the femur, tibia and patella; at least one sensor communicating with the plurality of microsensors; a navigation system coupled to the at least one sensor; an imaging system coupled to the navigation system for performing imaging of the joint; and at least one display for displaying imaging and tracking data.

### CROSS-REFERENCE TO RELATED APPLICATIONS

This application claims priority to (is based on and claims the benefit of) U.S. Provisional Patent Application No. 60/864,748, filed Nov. 7, 2006, the disclosure of which is incorporated herein by reference.

### BACKGROUND OF THE INVENTION

The present invention generally relates to a system and method for measuring parameters of the knee, and more particularly to an intraoperative system and method for pre-incision measurement of biomechanical parameters of the knee for use during total knee replacement (TKR) surgery.

TKR surgery removes the damaged and painful areas of the femur (thigh bone), tibia (lower leg bone) and patella (kneecap). These areas are then replaced with specially designed metal and polyethylene prosthetic components.

During TKR surgery, damaged portions of the femur bone and cartilage are cut away. The end of the femur bone is reshaped to allow a metal femoral component to fit on the reshaped end of the femur bone. The metal femoral component is attached to the reshaped end of the femur bone with bone cement.

Also during TKR surgery, the damaged portions of the tibia bone and cartilage are cut away. The end of the tibia bone is reshaped to receive a metal tibial component. The metal tibial component is secured to the reshaped end of the tibia bone with bone cement. A polyethylene insert is attached to the top of the exposed end of the metal tibial component. The insert will support the body's weight and allow the femur to smoothly glide over the tibia, just like the tibia's original cartilage used to do. The tibia with its new polyethylene surface and the femur with its new metal component are put together to form a new knee joint.

To make sure that the patella glides smoothly over the new tibial component, the patella's rear surface is cut away and prepared to receive a polyethylene patellar component, which is cemented into place on the newly prepared rear surface of the patella with bone cement. The new parts of the knee joint are then tested by flexing and extending the knee.

As stated above, during TKR surgery, the surgeon uses prosthetic components that replace the ends of the tibia and femur, the underside of the patella and compensate for cartilage and some ligaments. The surgeon resurfaces the patella by using a saw within a mechanical jig to guide the cut. A polyethylene prosthetic patellar component is implanted on this smooth surface. After the surgery, the patellar component is in contact with a femoral component during each motion of the knee. Misplacement of the patellar component can increase wear of the polyethylene patellar component and possibly result in a fracture of the patella.

For proper alignment and pain free functioning of the prosthetic components, it is very important to know the size, shape, position and orientation of the original components of the knee. There are two main joints within the knee. The femorotibial joint and the patellofemoral joint. Currently, a surgeon does not have any tools to enable the precise alignment of the prosthetic components and joints during a TKR procedure.

It is very important to measure the clinical parameters of the knee before the surgical incision, including the size, shape and kinematics of the femur, tibia and patella. It is also important to know the size, shape and kinematics of the femorotibial and patellofemoral joints. Knowing this information prior to surgery would allow the surgeon to choose the proper implants and properly position and align the implants during surgery. Knowing the exact shape of the patella will allow the surgeon to realize a perfect resurfacing according to the thickness of the patella and position of the femoral component. This information will influence the distal femoral cut, without forgetting the anterior and posterior cuts. These last cuts defined the axial rotation of the femoral component. Also, knowing the exact trajectory of the patella along the femur will allow the surgeon to restore this trajectory by using the axial rotation of the femoral component (anterior and posterior cut), and the midline knee (height of the distal femoral cut plus height of the proximal tibial cut).

The problems to be solved include the inability to measure the size, shape and kinematics of the femur, tibia and patella prior to TKR surgery. The inability to measure the size, shape and kinematics of the femorotibial and patellofemoral joints prior to TKR surgery. This is due in part on the lack of available medical navigation sensors for measuring these clinical parameters and the lack of appropriate mounting techniques for the sensors. Generally, prior art tracking sensors are too large with ineffective and possibly damaging mounting techniques.

Therefore, there is a need for a system and method of measuring and analyzing the size, shape, position, orientation, and kinematics of knee components prior to TKR surgery to decrease the number of postoperative dislocations, fractures, and wear of implanted prosthetic components.

### BRIEF DESCRIPTION OF THE INVENTION

In an embodiment, a system for measuring biomechanical parameters of a joint comprising a plurality of microsensors removably attached to bones of the joint; at least one sensor communicating with the plurality of microsensors; a navigation system coupled to the at least one sensor; an imaging system coupled to the navigation system for performing imaging of the joint; and at least one display for displaying imaging and tracking data.

In another embodiment, a system for measuring biomechanical parameters of an anatomical region of interest comprising a plurality of microsensors removably attached to the anatomical region of interest; at least one sensor communicating with the plurality of microsensors; an integrated imaging and navigation system coupled to the at least one sensor; and at least one display coupled to the integrated imaging and navigation system for displaying imaging and tracking data.

In yet another embodiment, a method for measuring biomechanical parameters of a joint comprising attaching a plurality of microsensors to bones of the joint using a minimally invasive procedure; imaging the joint with an imaging system; performing a first series of flexion and extension of the joint; tracking position and orientation of microsensors during the first series of flexion and extension; displaying imaging data and tracking data on a display; identifying areas of joint that need to be cut for optimal placement of implants; performing surgical incision, cutting of joint and implant placement; confirming alignment of original joint components with implants; performing a second series of flexion and extension of the joint; tracking position and orientation of microsensors during the second series of flexion and extension; confirming trajectory of original patella with trajectory of patellar implant during second series of flexion and extension; and removing the plurality of microsensors from the joint.

In still yet another embodiment, an intraoperative method for pre-incision measurement of biomechanical parameters of a patella of a knee undergoing total knee replacement surgery (TKR) comprising attaching a plurality of microsensors to the femur, tibia and patella; imaging the knee with an imaging system; performing a first series of flexion and extension of the knee; recording and storing position and orientation data of the femur, tibia and patella during the first series of flexion and extension; displaying imaging data of the knee, and position and orientation data of the femur, tibia and patella on a display; reviewing position and orientation data of the femur, tibia and patella with femur, tibia and patella parameters, and femoral, tibial and patellar component parameters to determine an optimal position for femoral, tibial and patellar component placement; identifying areas of the femur, tibia and patella that need to be cut for optimal placement of the femoral, tibial and patellar components; performing surgical incision, cutting of the femur, tibia and patella, and femoral, tibial and patellar component placement; confirming position and orientation of the femur, tibia and patella with the femoral, tibial and patellar components on the display; performing a second series of flexion and extension of the knee; confirming trajectory of the patella with trajectory of the patellar component during second series of flexion and extension; and removing the plurality of microsensors from the femur, tibia and patella.

Various other features, objects, and advantages of the invention will be made apparent to those skilled in the art from the accompanying drawings and detailed description thereof.

### BRIEF DESCRIPTION OF THE DRAWINGS

FIG. 1 is a diagram illustrating a knee with a plurality of microsensors attached to the bones of the knee according to an exemplary embodiment for measuring clinical parameters of the knee prior to total knee replacement (TKR) surgery;

FIG. 2 is a diagram illustrating an exemplary embodiment of a system for measuring clinical parameters of the knee during TKR surgery;

FIG. 3 is a flow diagram illustrating an exemplary embodiment of a method for measuring clinical parameters of the knee during TKR surgery;

FIG. 4 is a flow diagram illustrating another exemplary embodiment of a method for measuring clinical parameters of the knee during TKR surgery; and

FIG. 5 is a diagram illustrating a knee with a plurality of microsensors attached to the bones of the knee according to an exemplary embodiment for measuring clinical parameters of the knee after attaching implants during TKR surgery.

### DETAILED DESCRIPTION OF THE INVENTION

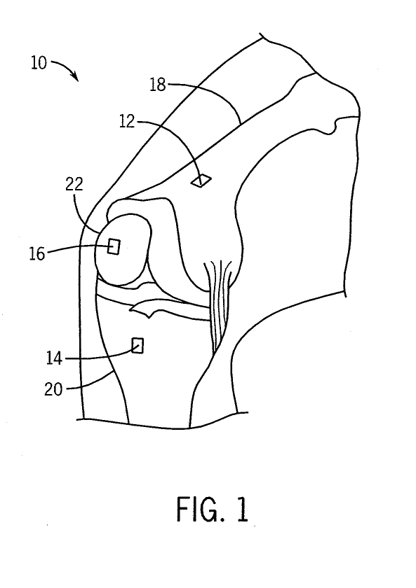

Referring to the drawings, FIG. 1 illustrates a preoperative knee 10 with three microsensors 12, 14, 16 attached to the femur 18, tibia 20, and patella 22. These microsensors 12, 14, 16 are part of a navigation system 36 used to track movement of the femur 18, tibia 20 and patella 22, and measure biomechanical parameters of the knee 10 prior to TKR surgery. The biomechanical parameters allow a surgeon to implant knee prostheses by taking into account the size, shape and movement of the femur 18, tibia 20 and patella 22, and to take into account femorotibial and patellofemoral kinematics.

The microsensors 12, 14, 16 are electromagnetic (EM) field generators that include microcoils for generating a magnetic field. At least one EM field sensor 34 is brought into proximity with the microsensors 12, 14, 16 to receive magnetic field measurements from the microsensors 12, 14, 16 for calculating the position and orientation of the microsensors 12, 14, 16. The microsensors 12, 14, 16 may be passively powered, powered by an external power source, or powered by an internal battery.

An exemplary method of attaching the microsensors 12, 14, 16 to the femur 18, tibia 20, and patella 22 is through a minimally invasive procedure using a bone biopsy needle (BBN) and a rigid guidewire. The procedure is performed with a patient lying on a surgical table prior to TKR surgery. This exemplary method includes making a hole in the patient's skin with the BBN; removing the inner part of BBN (needle); attaching a microsensor to the tip of a rigid guidewire; and inserting the rigid guidewire through the cannulated channel to attach the microsensor to the bone. The microsensors 12, 14, 16 are rigidly secured to the femur 18, tibia 20, and patella 22. The microsensors 12, 14, 16 will move with the femur 18, tibia 20, and patella 22 to provide kinematic information on these bones, and their corresponding femorotibial and patellofemoral joints. The size of the microsensors 12, 14, 16 are small enough that they do not affect movement of the femur 18, tibia 20, or patella 20, and do not modify the trajectory of the patella 20 during flexion and extension of the knee. The microsensors 12, 14, 16 are approximately 3.5 mm in diameter, or less, in order to avoid the risk of fracturing the patella, or affecting movement of the femur 18, tibia 20 or patella 22. After performing the TKR surgery, the microsensors 12, 14, 16 are removed from the femur 18, tibia 20, and patella 22.

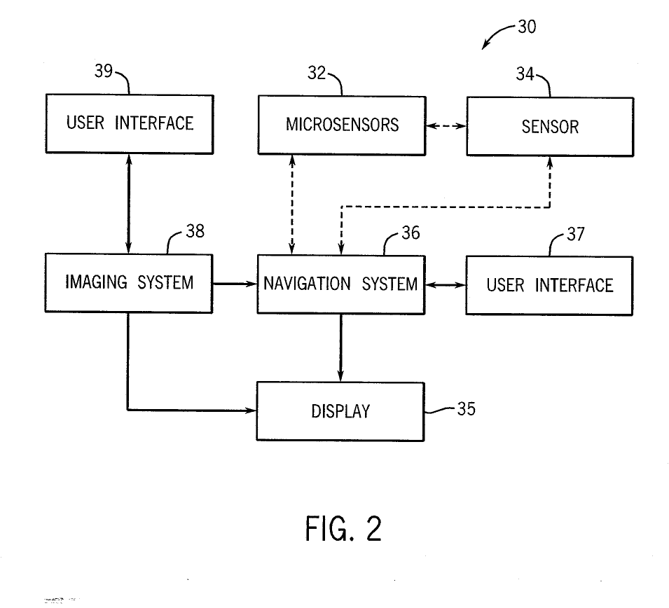

FIG. 2 is a diagram illustrating an exemplary embodiment of a system 30 for measuring biomechanical parameters of the knee during TKR surgery. These parameters allow a surgeon to more accurately place prostheses during surgery. The system 30 includes a plurality of microsensors 32 removably attached to the bones of the knee of a patient to be operated on, at least one sensor 34 external to the patient for communicating with and receiving data from the plurality of microsensors 32, a navigation system 36 coupled to and receiving data from the at least one sensor 34, an imaging system 38 coupled to the navigation system 36 for performing imaging of the knee, a first user interface 39 coupled to the imaging system 38, a second user interface 37 coupled to the navigation system 36, and a display 35 for displaying the imaging and tracking data. In another exemplary embodiment, the system 30 may only have one user interface coupled to both the imaging system 38 and the navigation system 36. In yet another exemplary embodiment, the imaging system 38 and the navigation system 36 may be integrated into a single system with integrated instrumentation and software.

The microsensors 32 enable a surgeon to continually track the position and orientation of the knee during surgery. After the plurality of microsensors 32 are attached to the bones of the knee, an EM field is generated around the microsensors 32. The at least one sensor 34 receives tracking data from the plurality of microsensors 32 attached to the knee that measure in real-time the passive motion of the knee during flexion and extension of the knee. The plurality of microsensors 32 are preferably EM field generators, and the at least one sensor 34 is preferably an EM field receiver. The EM field receiver may be a receiver array including at least one coil or at least one coil pair and electronics for digitizing magnetic field measurements detected by the receiver array. It should, however, be appreciated that according to alternate embodiments the microsensors 32 may be EM field receivers and the sensor 34 may be an EM field generator.

The magnetic field measurements can be used to calculate the position and orientation of the microsensors 32 according to any suitable method or system. After the magnetic field measurements are digitized using electronics on the sensor 34, the digitized signals are transmitted from the sensor 34 to the navigation system 36. The digitized signals may be transmitted from the sensor 34 to the navigation system 36 using wired or wireless communication protocols and interfaces. The digitized signals received by the navigation system 36 represent magnetic field information detected by the sensor 34. The digitized signals are used to calculate position and orientation information of the microsensors 32, including the location of the microsensors 32. The position and orientation information is used to register the location of the microsensors 32 to acquired imaging data from the imaging system 38. The position and orientation data is visualized on the display 38, showing in real-time the location of the microsensors 32 on pre-acquired or real-time images from the imaging system 38. The acquired imaging data from the imaging system 38 may include CT imaging data, MR imaging data, PET imaging data, ultrasound imaging data, X-ray imaging data, or any other suitable imaging data, as well as any combinations thereof. In addition to the acquired imaging data from various modalities, real-time imaging data from various real-time imaging modalities may also be available.

The navigation system 36 is configured to calculate the relative locations of the microsensors based on the received digitized signals. The navigation system further registers the location of the microsensors to the acquired imaging data, and generates imaging data suitable to visualize the image data and representations of the microsensors.

The navigation system 36 is illustrated conceptually and may be implemented using any combination of dedicated hardware boards, digital signal processors, field programmable gate arrays, and processors. Alternatively, the navigation system 36 may be implemented using an off-the-shelf computer with a single processor or multiple processors, with the functional operations distributed between processors. As an example, it may be desirable to have a dedicated processor for position and orientation calculations as well as a processor for visualization operations. The navigation system 36 is preferably an EM navigation system utilizing EM navigation technology. However, other tracking or navigation technologies may be used.

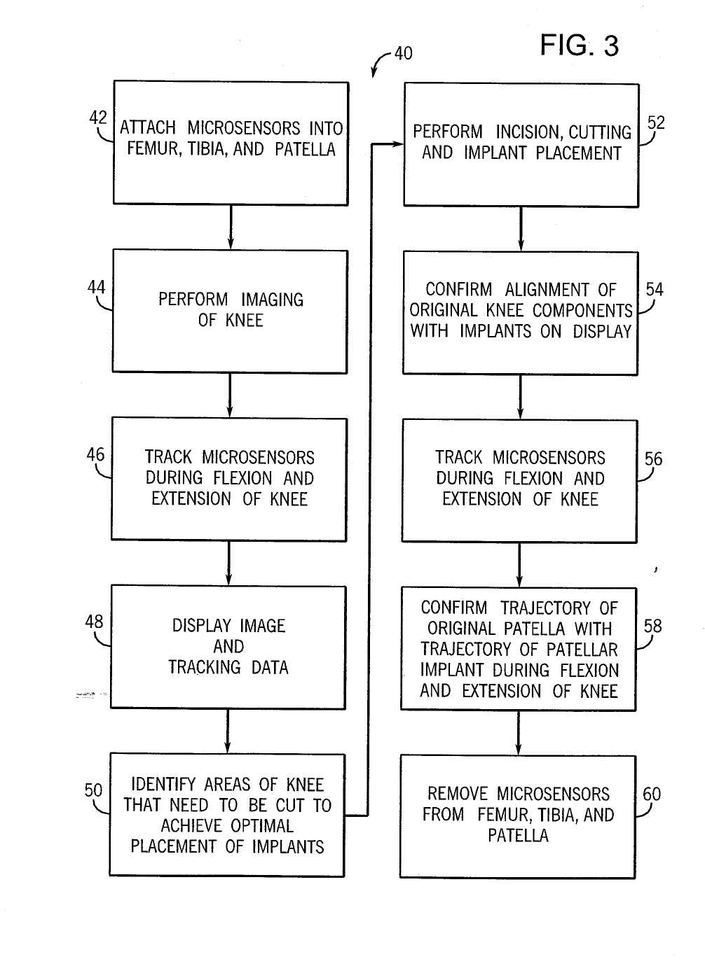

FIG. 3 is a flow diagram illustrating an exemplary embodiment of a method 40 for measuring clinical parameters of the knee during TKR surgery. The method includes removably attaching microsensors into the femur, tibia, and patella 42. Performing 3D imaging of the knee 44 using an imaging system. Tracking the microsensors during a first series of passive flexion and extension of the knee 46 to determine the position and orientation of the patella relative to the tibia and femur. The passive flexion and extension of the knee should be performed several times in order to achieve reproducible results as to the location and trajectory of the knee components during this procedure. The x, y, z coordinates of each microsensor during each flexion and extension of the knee are recorded and stored in memory. The method further includes displaying iconic representations of each bone (femur, tibia, patella) during flexion and extension, and superimposing the representations on a 3D registered image of the joint. The use of physical landmarks (microsensors) and kinematics provides real-time data on performing femoral and tibial cuts (location, slope, depth, angles). Another step in the process includes identify bony areas of the knee that need to be cut to achieve optimal placement of implants or prostheses 50. To ensure correct patellar component placement and alignment, the size, shape and kinematics of the patella and the size and shape of the patellar implant are taken into account. Another step includes performing the incision, cutting of damaged areas of the femur, tibia and patella, and attaching the implants 52. The navigation system allows a surgeon to navigate the proximal tibia cut (medial resection and lateral resection), and the distal femur cut (medial resection and lateral resection). The method further includes displaying a first iconic representation of the patella obtained during the first series of flexion and extension (this location is selected based on the relative position of the femur and tibia microsensors), and displaying with the first iconic representation, a second iconic representation of the patella with the patellar implant, representing the current position of the patella with the patellar implant, relative to first iconic representation of the original patella. The surgeon then confirms alignment of the first and second iconic representations that are superimposed on the displayed image 54. Another step includes tracking the microsensors during a second series of passive flexion and extension of the knee 56 to determine the position and orientation of the patellar implant relative to the tibia and femur implants. The trajectory of the patella from the first series of flexion and extension and the trajectory of the patellar implant from the second series of flexion and extension are displayed. The surgeon can then confirm the trajectory of the original patella with the trajectory of the patellar implant that are superimposed on the displayed image 58. The x, y and z coordinates of the patella and patellar implant should be the same, and movement of the patella and patellar implant within the patellofemoral joint should be the same as well. The final step is removing the microsensors from the femur, tibia, and patella 60.

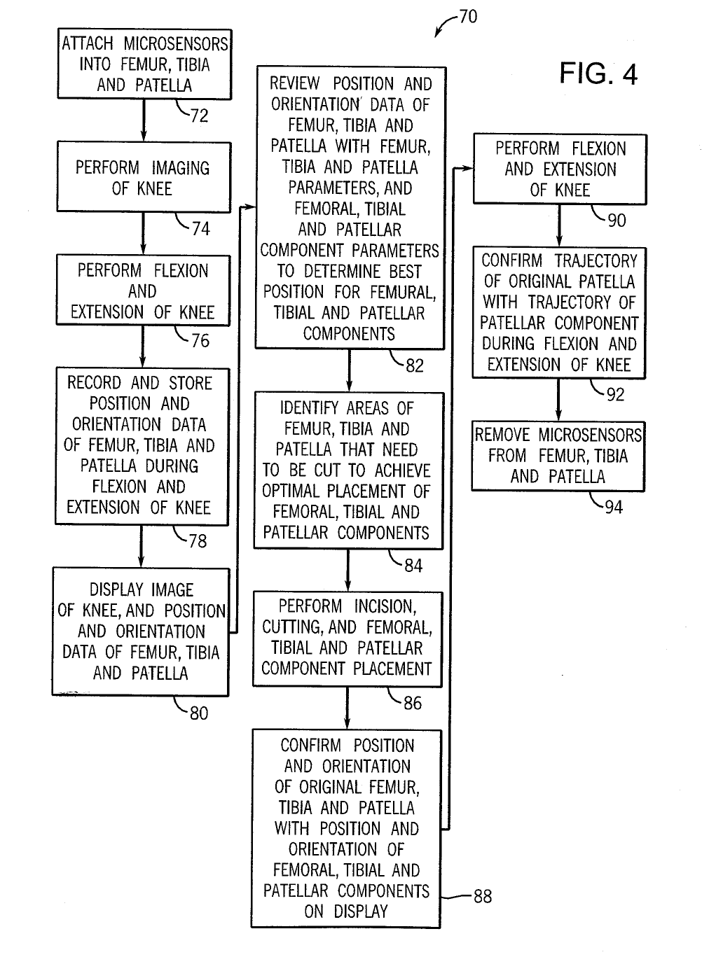

FIG. 4 is a flow diagram illustrating another exemplary embodiment of a method 70 for measuring clinical parameters of the knee during TKR surgery. The method includes removably attaching microsensors into the femur, tibia, and patella 72. Performing 3D imaging of the knee 74 using a 3D imaging system. A 3D reconstruction of the knee is performed to obtain a virtual representation of knee. The virtual representation of the knee is displayed on a display screen. The method further includes performing a first series of passive flexion and extension of the leg 76 to simulate knee motion. This passive flexion and extension of the knee should be performed several times in order to achieve reproducible results as to the location and trajectory of the knee components during this procedure. Another step is recording and storing the positions and orientations of the femur, tibia and patella in real time during flexion and extension of the knee 78. The x, y, z coordinates of each microsensor during each flexion and extension of the knee are recorded and stored in memory. In another step, the 3D reconstructed image of the knee and the position and orientation data of the femur, tibia and patella are displayed on the display 80. The surgeon can track the trajectory of the patella on the display screen by using the virtual representation of the femur, tibia and patella, and combining the kinematic data received during flexion and extension of the knee with current parameters (frontal and sagittal angulations) and the implant manufacturer's implant parameters to determine the best position for the tibial, femoral and patellar implant components 82. The method further includes identifying bony areas of the femur, tibia and patella that need to be cut to achieve optimal placement of the femoral, tibial and patellar components 84. To ensure correct patellar component placement and alignment, the size, shape and kinematics of the patella and the size and shape of the patellar implant are taken into account. Another step includes performing the incision, cutting of damaged areas of the femur, tibia and patella, and attaching the femoral, tibial and patellar components 86. The navigation system allows a surgeon to navigate the proximal tibia cut (medial resection and lateral resection), and the distal femur cut (medial resection and lateral resection). The method further includes displaying a first visual representation of the patella based on the positional information of the patella obtained during the first series of flexion and extension (this is determined based on the relative position of the femur and tibia microsensors), and displaying with the first visual representation a second virtual representation of the current position of the patellar implant relative to first visual representation. The surgeon then confirms alignment of the first and second virtual representations that are superimposed on the displayed image 88. Another step includes tracking the microsensors during a second series of passive flexion and extension of the knee 90 to determine the position of the patellar implant relative to the tibia and femur implants. The method further includes displaying the first trajectory of the patella from the first series of flexion and extension and the trajectory of the patellar implant from the second series of flexion and extension. The surgeon can then confirm the trajectory of the original patella with the trajectory of the patellar implant that are superimposed on the displayed image 92. The x, y and z coordinates of the patella should be the same, and movement of the patella and the patellar implant within the patellofemoral joint should be the same as well. The final step is removing the microsensors from the femur, tibia, and patella 94.

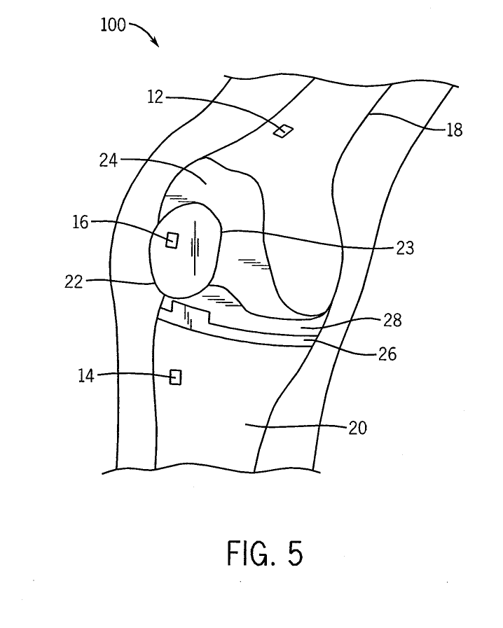

FIG. 5 illustrates a postoperative knee 100 with the three microsensors 12, 14, 16 attached to the femur 18, tibia 20, and patella 22. The microsensors 12, 14, 16 are part of the navigation system 36 used to track movement of the femur 18, tibia 20 and patella 22, and measure biomechanical parameters of the knee 10 during TKR surgery. In this figure, the surgeon has replaced the ends of the tibia and femur, and the underside of the patella with femoral, tibial, and patellar components. A femoral component 24 is attached to the reshaped end of the femur bone 18. A tibial component 26 is secured to the reshaped end of the tibia bone 20. An insert 28 is attached to the top of the exposed end of the tibial component 26. The insert supports the body's weight and allows the femur to smoothly glide over the tibia. A patellar component 23 is attached to the prepared rear surface of the patella 22. The patellar component 23 is in contact with a femoral component 24 during each motion of the knee. After the surgeon has confirmed correct placement of the femoral and tibial components, and the correct placement and trajectory of the patellar implant, the microsensors are removed from the femur, tibia and patella.

Although the proposed invention focuses on applying the benefits of imaging and tracking for knee replacement procedures that would provide simple workflow and high accuracy, it will be possible to extend this solution to other medical procedures.

Several embodiments are described above with reference to drawings. These drawings illustrate certain details of specific embodiments that implement the systems and methods and programs of the present invention. However, describing the invention with drawings should not be construed as imposing on the invention any limitations associated with features shown in the drawings. The present invention contemplates methods, systems and program products on any machine-readable media for accomplishing its operations. As noted above, the embodiments of the present invention may be implemented using an existing computer processor, or by a special purpose computer processor incorporated for this or another purpose or by a hardwired system.

As noted above, embodiments within the scope of the present invention include program products comprising machine-readable media for carrying or having machine-executable instructions or data structures stored thereon. Such machine-readable media can be any available media that can be accessed by a general purpose or special purpose computer or other machine with a processor. By way of example, such machine-readable media may comprise RAM, ROM, PROM, EPROM, EEPROM, Flash, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such a connection is properly termed a machine-readable medium. Combinations of the above are also included within the scope of machine-readable media. Machine-executable instructions comprise, for example, instructions and data which cause a general purpose computer, special purpose computer, or special purpose processing machines to perform a certain function or group of functions.

Embodiments of the invention are described in the general context of method steps which may be implemented in one embodiment by a program product including machine-executable instructions, such as program code, for example in the form of program modules executed by machines in networked environments. Generally, program modules include routines, programs, objects, components, data structures, etc. that perform particular tasks or implement particular abstract data types. Machine-executable instructions, associated data structures, and program modules represent examples of program code for executing steps of the methods disclosed herein. The particular sequence of such executable instructions or associated data structures represent examples of corresponding acts for implementing the functions described in such steps.

Embodiments of the present invention may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing the overall system or portions of the invention might include a general purpose computing device in the form of a computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated machine-readable media provide nonvolatile storage of machine-executable instructions, data structures, program modules and other data for the computer.

The foregoing description of embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principals of the invention and its practical application to enable one skilled in the art to utilize the invention in various embodiments and with various modifications as are suited to the particular use contemplated.

While the invention has been described with reference to various embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

What is claimed is:

1. A system for measuring biomechanical parameters of a joint comprising:
a plurality of microsensors removably attached to bones of the joint;
at least one sensor communicating with the plurality of microsensors;
a navigation system coupled to the at least one sensor;
an imaging system coupled to the navigation system for performing imaging of the joint; and
at least one display coupled to the imaging system and the navigation system for displaying imaging and tracking data;
wherein the imaging and tracking data includes a first set of kinematic parameters for the bones of the joint acquired during a first dynamic analysis of the bones of the joint prior to making an incision during surgery on the joint, and a second set of kinematic parameters of the bones of the joint acquired during a second dynamic analysis of the bones of the joint during placement of at least one bone implant on the bones of the joint during surgery; and
wherein the first and second kinematic parameters are compared to ensure proper placement of the at least one bone implant on the bones of the joint.

2. The system of claim 1, wherein the joint is the knee.

3. The system of claim 1, wherein the bones are the femur, tibia and patella.

4. The system of claim 3, wherein the plurality of microsensors are removably attached to the femur, tibia and the patella.

5. The system of claim 4, wherein the plurality of microsensors are small enough that they do not affect movement of the femur, tibia and the patella.

6. The system of claim 1, wherein the plurality of microsensors are electromagnetic (EM) field generators.

7. The system of claim 1, wherein the at least one sensor is an electromagnetic (EM) field receiver.

8. The system of claim 1, wherein the at least one sensor receives data from the plurality of microsensors.

9. The system of claim 1, wherein the navigation system receives data from the at least one sensor.

10. The system of claim 1, wherein the at least one display receives imaging data from the imaging system and receives tracking data from the navigation system.

11. A system for measuring biomechanical parameters of an anatomical region of interest comprising:
a plurality of microsensors removably attached to the anatomical region of interest;
at least one sensor communicating with the plurality of microsensors;
an integrated imaging and navigation system coupled to the at least one sensor; and
at least one display coupled to the integrated imaging and navigation system for displaying imaging and tracking data;
wherein the imaging and tracking data are used for tracking the motion of the anatomical region of interest.

12. A method for measuring biomechanical parameters of a joint comprising:
attaching a plurality of microsensors to bones of the joint using a minimally invasive procedure;
imaging the joint with an imaging system;
performing a first dynamic analysis of the joint;
tracking position and orientation of microsensors during the first dynamic analysis;
displaying imaging data and tracking data on a display;
identifying areas of joint that need to be cut for optimal placement of implants;
performing surgical incision, cutting of joint and implant placement;
confirming alignment of original joint components with implants;
performing a second dynamic analysis of the joint;
tracking position and orientation of microsensors during the second dynamic analysis;
confirming trajectory of original joint components with trajectory of implants during the second dynamic analysis; and
removing the plurality of microsensors from the bones of the joint.

13. The method of claim 12, wherein the joint is the knee.

14. The method of claim 12, wherein the bones are the femur, tibia and patella.

15. The method of claim 12, wherein the imaging step provides 3D imaging of the joint.

16. The method of claim 14, wherein the first tracking step determines the position and orientation of the patella relative to the femur and tibia.

17. The method of claim 14, wherein the first tracking step includes measuring the size, shape and kinematics of the patella.

18. The method of claim 17, wherein the first tracking step further includes determining the trajectory of the patella within the patellofemoral joint.

19. The method of claim 12, wherein the displaying imaging data and tracking data step includes displaying iconic representations of each bone during flexion and extension, and superimposing the representations on a 3D registered image of the joint.

20. The method of claim 19, further comprising the step of displaying a first iconic representation of the patella obtained during the first flexion and extension, and displaying with the first iconic representation, a second iconic representation of the patella with the patellar implant, representing the current position of the patella with patellar implant, relative to first iconic representation.

21. An intraoperative method for pre-incision measurement of biomechanical parameters of a patella of a knee undergoing total knee replacement surgery (TKR) comprising:
attaching a plurality of microsensors to the femur, tibia and patella;
imaging the knee with an imaging system;
performing a first series of flexion and extension of the knee;
recording and storing position and orientation data of the femur, tibia and patella during the first series of flexion and extension;
displaying imaging data of the knee, and position and orientation data of the femur, tibia and patella on a display;
reviewing position and orientation data of the femur, tibia and patella with femur, tibia and patella parameters, and femoral, tibial and patellar component parameters to determine an optimal position for femoral, tibial and patellar component placement;
identifying areas of the femur, tibia and patella that need to be cut for optimal placement of the femoral, tibial and patellar components;
performing surgical incision, cutting of the femur, tibia and patella, and femoral, tibial and patellar component placement;
confirming position and orientation of the femur, tibia and patella with the femoral, tibial and patellar components on the display;
performing a second series of flexion and extension of the knee;
confirming trajectory of the patella with trajectory of the patellar component during second series of flexion and extension; and
removing the plurality of microsensors from the femur, tibia and patella.
